SEQUENCE LISTING (18 sequences)

SEQ ID NO: 1 - protein, 787 aa, Ustilago maydis
MVKGSSNRRQHSASLQGLPSSQHCAPVISIPSPPPPAEDHAYPPSSFTIPLSKDEELAEAGPSRPGSSAISRRPVLSRRRTSKKEYVHPYLSGNFAPVTTECPLTDCLFEGTIPEEFAGSQYVRNGGNPLANSERDRDAHWFDADGMLAGVLFRRTPKGTIQPCFLNRFILTDLLLSTPEHSRLPYVPSIATLVNPHTSVFWLLCEIIRTFVLAMLTWLPGLGLGGNQKLKRISVANTSVFWHDGKAMAGCESGPPMRIMLPGLETAGWYTGEEDKEKETCDKNSGNSLTSSSSKGFGGGPPIVSMLREFTTAHPKIDPRTQELLLYHMCFEPPYLRISVIPASQSKKTDLPAHAKTIKGKAVRGLKQPKMMHDFGATATQTVIIDVPLSLDMMNLVRGKPILHYDPSQPTRFGILPRYEPERVRWYESAEACCIYHTANSWDDDGKFDASHEHATRSAIRGVNMLGCRLNSATLVYSAGNLLPPSHVLPPPNCPEKCQLYYWRFDLEHAETNTISHEFALSDIPFEFPTINEDYSMQQACYVYGTSMRDGTFDAGLGKAAKIDALVKLDAQALIRKGKAMWSQGRLKAGDSVDTRTVEEVLTAQRDGSASPEDPIKIFEMPRGWYAQETTFVPRRSSTNETSQEDDGWLVCYVFDEATGLHPSTGEVLPGASSELWIIDAKLMSRVVCRIKLPQRVPYGLHGTLFTEEQIASQKPIDPSQVRSWALSINLADPFSSSALGSTVYSAAGKAATSKFKNREETYAAFIKDPIRIGAWWVKRNIELLIA

SEQ ID NO: 2 - DNA, 2364 bp, Artificial Sequence
  Yarrowia codon-optimized UmCCO1
atggttaagggctcctctaaccgacgacagcactccgcttcccttcagggactcccttcttctcagcactgtgcccccgttatctctattccttctccccctccccctgctgaggatcacgcttaccccccttcctctttcactattcctctctccaaggatgaggagcttgctgaggccggaccctctcgacccggttcctctgctatttctcgacgacctgttctgtctcgacgacgaacttctaagaaggagtacgttcacccctacctctccggcaactttgcccctgttaccactgagtgccctctcaccgattgtctctttgagggtactatccctgaggagtttgctggctcccagtacgtccgaaacggcggaaacccccttgccaactccgagcgagatcgagatgcccactggttcgatgctgacggtatgctggctggagttctctttcgacgaacccccaagggcaccattcagccttgtttcctcaaccgattcattctcaccgacctcctgctctctacccctgagcactctcgactcccttacgtcccttccatcgctactctcgtcaacccccacacttccgtcttttggctcctttgtgagatcatccgaactttcgttctggctatgcttacctggctccctggcctcggactcggtggcaaccagaagctcaagcgaatctctgttgctaacacctccgttttctggcacgacggaaaggctatggctggatgtgagtctggaccccctatgcgaatcatgctccctggtcttgagactgccggctggtacactggtgaggaggataaggagaaggagacttgtgataagaactctggcaactctctcacttcttcctcttctaagggttttggcggaggccctcccattgtctccatgcttcgagagtttaccactgctcaccccaagattgaccctcgaacccaggagctccttctctaccacatgtgcttcgagcccccttaccttcgaatctctgtcatccctgcttctcagtctaagaagactgacctccctgctcacgctaagaccattaagggtaaggctgtgcgaggtcttaagcagcccaagatgatgcacgatttcggcgctaccgccactcagaccgtcatcatcgacgtccctctctccctcgacatgatgaacctcgtccgaggcaagcccattctgcactacgatccctctcagcctacccgattcggtattcttccccgatacgagcctgagcgagtgcgatggtacgagtctgccgaggcttgctgtatctaccacaccgccaactcttgggatgacgatggcaagtttgacgcttctcacgagcacgctacccgatccgccatccgaggcgtcaacatgctcggctgccgactcaactctgccaccctcgtgtactctgctggaaaccttctccctccctctcacgtccttccccctcccaactgccctgagaagtgtcagctctactactggcgattcgaccttgagcacgctgagactaacaccatttcccacgagtttgctctgtccgacattcctttcgagttccccaccatcaacgaggactactctatgcagcaggcttgttacgtttacggtacttccatgcgagatggcacctttgacgctggactcggaaaggctgctaagattgacgcccttgttaagctggacgctcaggcccttattcgaaagggcaaggccatgtggtcccagggacgacttaaggctggagactctgtggacacccgaaccgttgaggaggttctcactgctcagcgagatggttctgcctcccctgaggaccctatcaagattttcgagatgccccgaggatggtacgctcaggagactaccttcgtccctcgacgatcctctactaacgagacttctcaggaggatgacggttggctcgtctgctacgtgttcgatgaggccactggccttcacccttccaccggagaggttctccctggcgcttcctccgagctgtggatcattgatgccaagctcatgtcccgagtcgtttgccgaatcaagctcccccagcgagtcccttacggactccacggcactctctttaccgaggagcagattgcctctcagaagcctatcgacccttctcaggtccgatcctgggctctgtctatcaaccttgccgatcccttctcctcttccgcccttggctctaccgtgtactccgccgctggtaaggctgccacctccaagtttaagaaccgagaggagacttacgctgccttcatcaaggaccctatccgaatcggcgcttggtgggtcaagcgaaacatcgagctcctgattgcttaa

SEQ ID NO: 3 - protein, 696 aa, Fusarium fujikuroi
MKFLQQNSFTQTSMSQPHEDVSPAIRHPYLTGNFAPIHKTTNLTPCTYSGCIPPELTGGQYVRNGGNPVSHQDLGKDAHWFDGDGMLSGVAFRKASIDGKTIPEFVNQYILTDLYLSRKTTSIASPIMPSITTLVNPLSTMFQIMFATFRTIFLVILSNLPGSQQAIKRISVANTAVLYHDGRALATCESGPPMRIQLPSLDTVGWFDGVEAEGEPEISQAGSDDSPFGGSGIFSFMKEWTTGHPKVDPVTGEMLLYHNTFMPPYVHCSVLPKSNEKAPGHRLVNQPVLGVSGARMMHDFGASRSHTIIMDLPLSLDPLNTMKGKEVVAYDPTKPSRFGVFPRHLPSSVRWFHTAPCCIFHTANTWDSQSSEGELSVNLLACRMTSSTLVYTAGNIRPPVRSRCTQARVWSDEREETACRYKEAPALESPGESTGLADYFPITAESDDYDQCRLYYYEFDLAMESRNHVKSQWALSAIPFEFPSVRPDREMQEARYIYGCSTSTSCFGVALGRADKVDLLVKMDAKTLIQRGKKMNATSITGCVDRRSVCEILQEQRKDDPIYIFRLPPNHYAQEPRFVPRACSTEEDDGYLLFYVFDESQLLPSGDCPPSATSELWILDAKNMRDVVAKVRLPQRVPYGLHGTWFSSQDIESQRSVESLRSLEVVQRKKEEWVNSGGQIRKSWMVLREKLEKAVG

SEQ ID NO: 4 - DNA, 2091 bp, Artificial Sequence
  Yarrowia codon-optimized FfCarX
atgaagtttctccagcagaactcctttacccagacctctatgtctcagcctcacgaggatgtctctcccgccattcgacacccttaccttaccggcaactttgctcctattcacaagaccactaacctcactccctgtacttactctggctgcattccccccgagcttaccggaggtcagtacgttcgaaacggcggaaaccctgtctcccaccaggatctcggaaaggatgctcactggttcgatggcgacggtatgctctctggcgtcgcctttcgaaaggcttccattgatggcaagactatccctgagttcgttaaccagtacattcttaccgacctttacctttctcgaaagaccacctctattgcttcccctattatgccctctatcaccaccctggttaaccctctctctactatgtttcagatcatgttcgccaccttccgaactatcttcctcgtcattctctccaacctccctggttctcagcaggctatcaagcgaatctccgttgccaacactgctgttctttaccacgatggtcgagctcttgccacttgcgagtctggcccccccatgcgaatccagcttccctccctcgataccgttggctggttcgacggtgttgaggctgagggtgagcctgagatttctcaggccggctctgatgactctcccttcggcggttccggcatcttctcctttatgaaggagtggaccaccggccaccctaaggtggaccccgttaccggagagatgcttctctaccacaacaccttcatgcctccctacgtgcactgctctgttcttcccaagtctaacgagaaggctcccggacaccgacttgttaaccagcccgttcttggtgtttctggtgcccgaatgatgcacgacttcggagcctctcgatctcacactatcatcatggaccttcccctgtctctggaccctctcaacactatgaagggaaaggaggttgttgcttacgaccctaccaagccttctcgattcggtgtgttcccccgacaccttccctcttccgtgcgatggtttcacactgctccttgctgtatctttcacactgctaacacttgggattctcagtcctctgagggagagctttctgttaacctccttgcctgccgaatgacctcttctacccttgtttacactgccggcaacatccgacctcccgttcgatctcgatgtactcaggcccgagtctggtccgatgagcgagaggagactgcttgtcgatacaaggaggctcctgctcttgagtctcctggtgagtccactggccttgccgactactttcccattaccgctgagtccgacgactacgatcagtgccgactctactactacgagtttgaccttgctatggagtcccgaaaccacgtcaagtcccagtgggctctctctgccattcctttcgagtttccctctgtgcgacctgaccgagagatgcaggaggctcgatacatctacggctgttccacttccacttcttgcttcggtgtggctctcggacgagctgataaggttgaccttctcgttaagatggatgccaagaccctcattcagcgaggaaagaagatgaacgctacttccatcaccggatgcgttgatcgacgatctgtctgcgagatccttcaggagcagcgaaaggatgaccctatttacattttccgacttccccctaaccactacgctcaggagccccgattcgttccccgagcttgttctactgaggaggacgacggatacctccttttctacgtgttcgacgagtctcagctccttccctctggcgattgtcctccctctgctacttctgagctttggattcttgacgctaagaacatgcgagatgttgtggccaaggtccgacttccccagcgagttccttacggtctgcacggtacttggttctcttctcaggatattgagtctcagcgatctgtggagtctcttcgatctcttgaggttgtgcagcgaaagaaggaggagtgggttaactctggaggccagattcgaaagtcctggatggttcttcgagagaagctggagaaggctgttggatag

SEQ ID NO: 5 - protein, 369 aa, Crocus sativus
MQVDPTKGIGLANTSLQFSNGRLHALCEYDLPYVVRLSPEDGDISTVGRIENNVSTKSTTAHPKTDPVTGETFSFSYGPIQPYVTYSRYDCDGKKSGPDVPIFSFKEPSFVHDFAITEHYAVFPDIQIVMKPAEIVRGRRMIGPDLEKVPRLGLLPRYATSDSEMRWFDVPGFNMVHVVNAWEEEGGEVVVIVAPNVSPIENAIDRFDLLHVSVEMARIELKSGSVSRTLLSAENLDFGVIHRGYSGRKSRYAYLGVGDPMPKIRGVVKVDFELAGRGECVVARREFGVGCFGGEPFFVPASSKKSGGEEDDGYVVSYLHDEGKGESSFVVMDARSPELEILAEVVLPRRVPYGFHGLFVTEAELLSQQ

SEQ ID NO: 6 - DNA, 1110 bp, Artificial Sequence
  Yarrowia codon-optimized CsZCO
atgcaggtggaccccaccaagggtatcggcctggccaacacttctctccagttctccaacggacgactccacgctctttgcgagtacgacctcccctacgtcgttcgactctcccccgaggacggtgacatctctaccgtcggacgaatcgagaacaacgtttctactaagtctaccaccgcccaccccaagaccgaccccgtcaccggagagaccttctctttctcctacggtcccattcagccctacgtcacctactcccgatacgactgcgacggcaagaagtccggccccgacgtgcccatcttctctttcaaggagccctctttcgtccacgacttcgccatcaccgagcactacgccgtctttcccgacattcagatcgtgatgaagcccgccgagatcgttcgaggacgacgaatgatcggccccgaccttgagaaggtcccccgactgggccttctcccccgatacgccacctccgactccgagatgcgatggttcgacgtgcccggtttcaacatggttcacgtggttaacgcttgggaggaggagggcggagaggtcgtggtcatcgtggcccccaacgtgtcccccattgagaacgccatcgaccgattcgacctcctccacgtgtctgtggagatggcccgaatcgagctgaagtccggttccgtgtcccgaacccttctctctgccgagaacctcgatttcggtgtgattcaccgaggctactccggtcgaaagtcccgatacgcttacctcggagtcggcgaccccatgcccaagattcgaggtgtggtcaaggtggacttcgagctggccggacgaggagagtgcgtggttgcccgacgagagttcggcgtgggttgtttcggtggagagcccttctttgtccccgcttcttccaagaagtctggaggcgaggaggacgatggctacgttgtgtcttaccttcacgacgagggaaagggagagtcctctttcgtcgtgatggacgctcgatctcccgagctggagattcttgccgaggtggttctgccccgacgagttccctacggttttcacggcctctttgttaccgaggccgagcttctctcccagcagtag

SEQ ID NO: 7 - protein, 620 aa, Drosophila melanogaster
MAAGVFKSFMRDFFAVKYDEQRNDPQAERLDGNGRLYPNCSSDVWLRSCEREIVDPIEGHHSGHIPKWICGSLLRNGPGSWKVGDMTFGHLFDCSALLHRFAIRNGRVTYQNRFVDTETLRKNRSAQRIVVTEFGTAAVPDPCHSIFDRFAAIFRPDSGTDNSMISIYPFGDQYYTFTETPFMHRINPCTLATEARICTTDFVGVVNHTSHPHVLPSGTVYNLGTTMTRSGPAYTILSFPHGEQMFEDAHVVATLPCRWKLHPGYMHTFGLTDHYFVIVEQPLSVSLTEYIKAQLGGQNLSACLKWFEDRPTLFHLIDRVSGKLVQTYESEAFFYLHIINCFERDGHVVVDICSYRNPEMINCMYLEAIANMQTNPNYATLFRGRPLRFVLPLGTIPPASIAKRGLVKSFSLAGLSAPQVSRTMKHSVSQYADITYMPTNGKQATAGEESPKRDAKRGRYEEENLVNLVTMEGSQAEAFQGTNGIQLRPEMLCDWGCETPRIYYERYMGKNYRYFYAISSDVDAVNPGTLIKVDVWNKSCLTWCEENVYPSEPIFVPSPDPKSEDDGVILASMVLGGLNDRYVGLIVLCAKTMTELGRCDFHTNGPVPKCLHGWFAPNAI

SEQ ID NO: 8 - DNA, 1863 bp, Artificial Sequence
  Yarrowia codon-optimized DmNinaB
atggccgctggtgttttcaagtcttttatgcgagatttctttgctgttaagtacgatgagcagcgaaacgacccccaggccgagcgactggacggcaacggacgactgtaccccaactgctcctctgatgtttggcttcgatcttgcgagcgagagatcgttgaccccattgagggccaccactccggtcacattcccaagtggatttgcggttccctgctccgaaacggccccggctcttggaaggttggcgacatgaccttcggccacctgttcgactgctccgccctgctccaccgatttgccattcgaaacggacgagtcacctaccagaaccgatttgttgacactgagactctgcgaaagaaccgatctgcccagcgaattgttgtcaccgagtttggcactgccgctgttcccgatccctgtcactccatcttcgaccgatttgccgccatttttcgacccgattctggaaccgataactccatgatttccatctaccccttcggcgaccagtactacactttcaccgagactccctttatgcaccgaattaacccctgcactctcgctactgaggctcgaatctgcaccaccgacttcgttggcgttgtcaaccacacttctcacccccacgttcttccctctggcactgtttacaacctgggcaccactatgacccgatctggacccgcttacactatcctctctttcccccacggcgagcagatgttcgaggacgctcacgttgtcgccactctgccctgccgatggaagctgcaccccggttatatgcacaccttcggcctcactgaccactactttgtcattgttgagcagcccctttccgtttccctcactgagtacatcaaggcccagcttggcggacagaacctttccgcttgcctcaagtggttcgaggaccgacccactctctttcaccttattgatcgagtttccggcaagctggtccagacctacgagtccgaggctttcttctacctgcacatcatcaactgctttgagcgagatggccacgttgtcgttgacatttgctcttaccgaaaccccgagatgattaactgcatgtacctggaggccattgccaacatgcagactaaccccaactacgctaccctctttcgaggacgaccccttcgattcgtcctgcccctcggcactattccccccgcctctatcgccaagcgaggactcgtcaagtccttctccctcgctggactctccgctccccaggtttctcgaaccatgaagcactccgtttctcagtacgccgatattacctacatgcccaccaacggaaagcaggccactgctggagaggagtcccccaagcgagatgccaagcgaggccgatacgaggaggagaaccttgtcaacctggttactatggagggctctcaggccgaggcttttcagggcaccaacggcattcagcttcgacccgagatgctgtgtgattggggctgtgagactccccgaatctactacgagcgatacatgggcaagaactaccgatacttctacgccatttcttccgatgttgatgctgtcaaccccggcaccctcatcaaggttgatgtctggaacaagtcttgtcttacctggtgcgaggagaacgtctacccctctgagcccatttttgtcccctctcccgatcccaagtccgaggacgatggcgttatcctggcctctatggttcttggcggtcttaacgaccgatacgtcggccttattgttctttgtgccaagaccatgaccgagctgggccgatgtgatttccacaccaacggacccgttcccaagtgcctccacggttggtttgctcccaacgccatttag

SEQ ID NO: 9 - protein, 525 aa, Danio rerio
MLSFFWRNGIETPEPLKADVSGSIPPWLQGTLLRNGPGLFSVGNTSYKHWFDGMALIHSFTFKDGEVFYRSKYLKSETYKKNIAADRIVVSEFGTMVYPDPCKNIFSRAFSYMMNAIPDFTDNNLINIIKYGEDYYASSEVNYINQIDPLTLETLGRTNYRNHIAINLATAHPHYDEEGNTYNMGTAIMNLGRPKYVIFKVPANTSDKENKKPALSEVEQVCSIPIRPSLYPSYFHSFGMTENYIIFVEQAFKLDIVKLATAYFRDINWGSCLKFDQDDINVFHLVNKKTGKAVSVKYYTDPFVTFHHINAYEDDGHVVFDLITYKDSKLYDMFYIQNMKQDVKRFIETNKDFAQPVCQRFVLPVNVDKETPQDINLVKLQDTTATAVLKEDGSVYCTPDIIFKGLELPAINYKFNSKKNRYFYGTRVEWSPYPNKVAKVDVVTRTHKIWTEEECYPSEPVFIASPDAVDEDDGVILSSVVSFNPQRPPFLVVLDAKSFKEIARATIDASIHMDLHGLFIHDKST

SEQ ID NO: 10 - DNA, 1578 bp, Artificial Sequence
  Yarrowia codon-optimized DrBCO
atgctctctttcttctggcgaaacggtatcgagacccccgagcccctcaaggctgacgtttccggctctatccctccctggcttcagggaacccttctccgaaacggtcctggtctgttctccgttggcaacacttcctacaagcactggttcgatggtatggctctcattcactccttcacctttaaggatggtgaggttttttaccgatctaagtacctgaagtctgagacttacaagaagaacatcgctgccgaccgaatcgttgtgtctgagttcggaaccatggtgtaccccgatccctgcaagaacattttctcccgagccttctcttacatgatgaacgccattcctgactttaccgataacaacctcattaacatcattaagtacggtgaggattactacgcctcctctgaggtcaactacatcaaccagattgaccccctgacccttgagactctcggacgaactaactaccgaaaccacattgccatcaaccttgccactgctcaccctcactacgacgaggagggtaacacttacaacatgggcactgctattatgaacctcggtcgacccaagtacgtgattttcaaggtgcccgccaacacctctgataaggagaacaagaagcctgccctctctgaggtggagcaggtttgctccattcccatccgaccctccctttacccttcttacttccactcttttggcatgactgagaactacatcatcttcgttgagcaggccttcaagctggacatcgtcaagctggctactgcttacttccgagatattaactggggatcttgccttaagttcgaccaggatgacattaacgtgttccacctggtcaacaagaagactggtaaggctgtgtccgtgaagtactacactgacccctttgttaccttccaccacatcaacgcttacgaggacgatggccacgtcgtcttcgatctcattacttacaaggactctaagctgtacgatatgttctacattcagaacatgaagcaggacgtcaagcgatttattgagactaacaaggacttcgctcagcccgtgtgccagcgatttgtccttcccgtcaacgttgataaggagacccctcaggacatcaaccttgtcaagctgcaggacaccactgccactgctgtcctgaaggaggacggctctgtctactgcacccctgacatcatttttaagggtcttgagctccctgctatcaactacaagtttaactctaagaagaaccgatacttctacggcacccgagtggagtggtccccttaccctaacaaggtcgctaaggtggacgttgttactcgaacccacaagatttggactgaggaggagtgttacccttctgagcctgtctttattgcctcccctgacgccgttgatgaggatgacggtgtgattctttcttctgtggtttctttcaacccccagcgaccccctttcctggttgtcctcgatgctaagtccttcaaggagattgctcgagctaccatcgatgcctctattcacatggaccttcacggccttttcatccacgacaagtctacctaa

SEQ ID NO: 11 - protein, 281 aa, Artificial Sequence
  Danio rerio BCO TPI aa
KQKSNHILQYSPVITASITPVQVSLGFLFTDTVIYLTISLQVTQKVHVGNEPQTKTRYDKIALFDAEFDGVSIGVMTFICIHTKKSWWYFCVITSDIYAPPNPPATVKSVSLLYMLTKPPTVQRNPSAKSHNQLITTHPMTSPQILYAFRHYYSSLQRRCLRFHFCSITSLNPYRQIRPWHVSRLISPRVLHQGGGVRNTVRAHSKGVRVRASDNIAWTRRHILDFWARCIHLLRFPTLPPVSPSQPIEGNLIRDTFVIHSQIYKQCHSPSYSYIQHNYIQ

SEQ ID NO: 12 - DNA, 880 bp, Artificial Sequence
  Yarrowia codon-optimized DrBCO-TPI
aaacaaaagagctgaaatcatatccttcagtagtagtatagtcctgttatcacagcatcaattacccccgtccaagtaagttgattgggatttttgtttacagatacagtaatatacttgactatttctttacaggtgactcagaaagtgcatgttggaaatgagccacagaccaagacaagatatgacaaaattgcactattcgatgcagaattcgacggtgtttccattggtgttatgacattcatctgcattcatacaaaaaagtcttggtagtggtacttttgcgttattacctccgatatctacgcaccccccaacccccctgctacagtaaagagtgtgagtctactgtacatgcttactaaaccacctactgtacagcgaaacccctcagcaaaatcacacaatcagctcattacaacacacccaatgacctcaccacaaattctatacgccttttgacgccattattacagtagcttgcaacgccgttgtcttaggttccatttttagtgctctattacctcacttaacccgtataggcagatcaggccatggcactaagtgtagagctagaggttgatatcgccacgagtgctccatcagggctagggtggggttagaaatacagtccgtgcgcactcaaaaggcgtccgggttagggcatccgataatatcgcctggactcggcgccatattctcgacttctgggcgcgttgtattcatctcctccgcttcccaacacttccacccgtttctccatcccaaccaatagaatagggtaaccttattcgggacactttcgtcatacatagtcagatatacaagcaatgtcactctccttcgtactcgtacatacaacacaactacattcaaa

SEQ ID NO: 13 - protein, 531 aa, Ictalurus punctatus
MEAIFCRNGTETPEPVKAVVSGAIPPWLQGTLLRNGPGLFSIGKTSYNHWFDGLSLIHSFTFKHGDVYYRSKFLRSDTYKKNIAANRIVVSEFGTMVYPDPCKNIFSKAFTYLLNSIPDFTDNNLVSIIKYGDDYYTSSEINYINQINPVTLDTIGRANYRNYISLNLATAHPHYDDEGNTYNMGTAILAMSGPKYVIFKVPATTSDIKDNGKTNLALKNLQQICAIPFRSKLYPSYYHSFGMTQNYIIFVEQPFKLDIIRLATAYFRRTTWGKCLFYDQDDVTLFHIINRKTGDAVNTKFYGDALVVFHHINAYEEDGHIVFDLISYKDSSLYDLFYIDYMKQEAPKFTETSKAFSRPVCQRFVIPLNADLKGNPLGKNLVRLEDTSATAVFQMDGSLYCTPETLFQGLELPSINYQYNGKKYRYFYGSMMDWSPQANKIAKVDVDTKTHLEWTEEDCYPSEPKFVASPGAVDEDNGVILSSVVSVNPKKSPFMLVLDAKTLKEIARASIDATVHLDLHGIFIPQETELK

SEQ ID NO: 14 - DNA, 1596 bp, Artificial Sequence
  Yarrowia codon-optimized IpBCO
atggaggccattttctgtcgaaacggcaccgagactcccgagcccgtcaaggctgttgtgtccggtgctatccccccttggcttcagggaacccttctccgaaacggacccggccttttctccattggtaagacttcctacaaccactggtttgacggactctctcttattcactctttcacctttaagcacggtgatgtttactaccgatctaagttcctccgatccgatacctacaagaagaacattgctgccaaccgaatcgttgtgtctgagtttggcactatggtctaccccgatccctgcaagaacattttctctaaggccttcacttacctgctcaactctattcccgatttcaccgacaacaaccttgtctctattattaagtacggcgatgactactacacttcttccgagattaactacatcaaccagatcaaccccgttactctcgacactattggacgagccaactaccgaaactacatttcccttaaccttgctactgcccaccctcactacgatgacgagggaaacacctacaacatgggcactgctatcctggctatgtctggacccaagtacgtcatcttcaaggtgcccgctactacctctgatattaaggacaacggaaagactaaccttgctctgaagaacctgcagcagatctgcgccattcctttccgatctaagctctacccttcttactaccactcctttggtatgactcagaactacatcattttcgttgagcagcccttcaagctggacattattcgactggccactgcttacttccgacgaaccacctggggcaagtgcctcttttacgaccaggacgatgttactctcttccacattatcaaccgaaagactggtgacgccgtgaacactaagttctacggtgatgctctcgtggttttccaccacatcaacgcctacgaggaggacggccacatcgtttttgacctgatctcttacaaggactcttctctctacgaccttttctacattgactacatgaagcaggaggctcctaagttcactgagacttccaaggctttttctcgacccgtctgtcagcgattcgtcatccctctcaacgctgacctcaagggaaaccccctgggcaagaaccttgtccgacttgaggacacttctgctaccgctgtgttccagatggacggttccctgtactgtactcccgagactctctttcagggtcttgagctcccttccattaactaccagtacaacggaaagaagtaccgatacttctacggctctatgatggattggtcccctcaggctaacaagatcgctaaggtggacgttgataccaagactcaccttgagtggaccgaggaggattgctacccttctgagcctaagtttgtcgcttcccctggcgctgtcgatgaggataacggtgtgatcctgtcttctgttgtctccgtcaaccccaagaagtccccctttatgctcgtgctcgatgctaagaccctcaaggagatcgctcgagcctctattgacgccactgttcacctcgacctccacggaattttcatccctcaggagactgagcttaagtaa

SEQ ID NO: 15 - protein, 527 aa, Esox lucius
MAQIIFGKNGTESPEPVKAEITGCIPEWLQGTLLRNGPGLFKVGDTEYNHWFDGMALIHSFTFKDGDVYYRSKFLRSDTFQKNTKANKIVVSEFGTMIYPDPCKNMFSKAFSYLLAAIPDFTDNNLINIIRYGEDYYASSEINYINQIDPVTLEVIGKMNYRKHISLNLATAHPHYDEEGNTYNMGIALMRFGMPKYVIFKVPVDASDKEGKKPALEEVEQVCNIPFRSTLFPSYFHSFGMSENYIIFVEQPFKLDILRLATANFRGSTWGSCLKYDKEDITLIHLVDKKTGKAVSTKFYADALVVFHHINAYEDDNHVVFDMITYKDSNLYEMFYLANMREESNKFIEDKVNFSQPICQRFVLPLNVDKDTTKGTNMVMLKNTTAKAVMQDDGSVYCKPDTIFAGLELPGINYKFNGKKYRYFYGSRVEWTPFPNKIGKVDILTKKHIEWTEEECYPSEPVFVASPGAMEEDDGVILSSIVSLNPNKSPFMLVLNAKNFEEIARASIDASVHLDLHGLFIPSQKTN

SEQ ID NO: 16 - DNA, 1584 bp, Artificial Sequence
  Yarrowia codon-optimized ElBCO
atggctcagattatttttggcaagaacggcactgagtctcctgagcctgtcaaggccgagattaccggatgtatccctgagtggctccagggtactctccttcgaaacggtcccggtcttttcaaggtgggtgataccgagtacaaccactggttcgatggcatggccctgattcactcttttaccttcaaggatggtgacgtgtactaccgatctaagttccttcgatccgacaccttccagaagaacactaaggctaacaagattgttgtgtctgagtttggcaccatgatttaccctgacccctgcaagaacatgttttccaaggctttctcctacctccttgctgccatccctgacttcaccgataacaacctgattaacattatccgatacggtgaggactactacgcctcttccgagatcaactacatcaaccagattgaccctgttaccctggaggtgattggaaagatgaactaccgaaagcacatttctctgaaccttgctactgcccaccctcactacgacgaggagggaaacacttacaacatgggaatcgccctcatgcgatttggcatgcccaagtacgtcatcttcaaggttcctgtcgatgcttctgataaggagggcaagaagcctgcccttgaggaggtggagcaggtctgcaacattccctttcgatctaccctcttcccctcttacttccactcttttggcatgtctgagaactacatcatctttgtcgagcagcctttcaagctggacatcctccgactggccactgctaacttccgaggatctacctggggttcctgcctgaagtacgacaaggaggacattactctcatccacctggtcgacaagaagactggtaaggctgtttccaccaagttctacgctgatgctctggttgttttccaccacattaacgcctacgaggacgacaaccacgtggttttcgatatgatcacctacaaggactccaacctgtacgagatgttctaccttgctaacatgcgagaggagtctaacaagttcattgaggacaaggtcaacttctcccagcctatctgccagcgatttgtcctccccctcaacgttgacaaggataccactaagggaaccaacatggtgatgctcaagaacactaccgccaaggccgtgatgcaggatgacggctctgtgtactgcaagcctgacaccatttttgctggtcttgagctccctggcattaactacaagttcaacggcaagaagtaccgatacttttacggctctcgagtggagtggactcccttccctaacaagattggaaaggtggacattctgaccaagaagcacattgagtggaccgaggaggagtgttacccctctgagcccgtttttgttgcctcccccggagctatggaggaggatgacggagtcattctttcttctattgtctctctcaaccctaacaagtcccccttcatgcttgtcctcaacgctaagaactttgaggagattgctcgagcctccatcgatgcctctgttcacctcgatctccacggactcttcattccctctcagaagactaactag

SEQ ID NO: 17 - protein, 531 aa, Latimeria chalumnae
MQSLFGKNKRECPEPIKAEVKGQIPAWLQGTLLRNGPGMHTVGETSYNHWFDGLALMHSFTFKDGEVFYQSKYLRSDTYKKNMEANRIVVSEFGTMAYPDPCKNIFSKAFSYLSHTIPEFTDNCLINIMKCGEDYYAVTETNFIRKIDPKSLDTLEKVDYTKYIALNLASSHPHYDAAGDTINMGTSIADKGKTKYLIVKIPNMKPVESEKKKKVYFKNLEVLCSIPSHGRLNPSYYHSFGITENYIVFVEQPFKLDLLKLATAYFRGINWASCLNFHSEDKTFIHIIDRRTKTSVSTKFHTDALVLYHHVNAYEEDGHVVFDVIAYNDSSLYDMFYLANVRQESAEFEAKNTSSSKPACRRFVIPLQPDKDAELGTNLVKLASTTADAIKEKDSIYCHPEILVEDIELPRINYNYNGKKYRYIYVTGIAWKPIPTKIVKFDTLTRKSVEWQEEDCWPAEPVFVPSPDAKEEDDGIVLSSIVCTSPNKFPFLLILDAKTFTELARASINADVHLDLHGYFIPEKKKAQITH

SEQ ID NO: 18 - DNA, 1596 bp, Artificial Sequence
  Yarrowia codon-optimized LcBCO
atgcagtctctgttcggtaagaacaagcgagagtgtcctgagcccattaaggctgaggtgaagggtcagattcctgcttggctccagggtactctccttcgaaacggccctggcatgcacaccgttggcgagacttcttacaaccactggttcgacggactcgctcttatgcactccttcacctttaaggatggtgaggttttttaccagtctaagtacctgcgatccgacacctacaagaagaacatggaggccaaccgaattgtcgtgtctgagttcggaaccatggcctaccccgatccctgcaagaacattttttccaaggctttttcttacctttctcacaccatccctgagtttaccgacaactgtctgatcaacattatgaagtgtggtgaggattactacgctgttactgagactaacttcatccgaaagattgatcccaagtccctcgacaccctggagaaggttgactacaccaagtacattgctcttaacctggcttcctcccacccccactacgatgctgctggtgataccattaacatgggcacctctatcgctgataagggaaagactaagtacctgattgttaagattcccaacatgaagcccgttgagtctgagaagaagaagaaggtctactttaagaacctggaggtgctctgctccatcccttctcacggacgacttaacccttcttactaccactcctttggcatcactgagaactacatcgttttcgtggagcagccctttaagctggaccttctcaagctggccaccgcctacttccgaggtattaactgggcctcttgtcttaacttccactccgaggacaagactttcattcacatcatcgatcgacgaaccaagacctccgtttccactaagtttcacaccgatgctctcgttctttaccaccacgtcaacgcttacgaggaggatggccacgttgttttcgatgtcattgcctacaacgactcttctctctacgatatgttttacctcgccaacgttcgacaggagtctgccgagtttgaggctaagaacacctcttcctccaagcctgcttgtcgacgatttgtcattcccctgcagcctgacaaggatgctgagctgggcactaacctggtcaagctcgcttccactaccgccgacgccattaaggagaaggactccatttactgccaccctgagatcctggttgaggatattgagctccctcgaattaactacaactacaacggcaagaagtaccgatacatttacgttactggtatcgcctggaagcccattcccactaagattgtcaagtttgacactctcactcgaaagtccgtggagtggcaggaggaggactgttggcccgccgagcctgtctttgttccttcccccgatgccaaggaggaggacgatggtattgttctttcttccatcgtgtgtacttcccctaacaagtttcccttcctccttattctggacgccaagacctttaccgagctcgctcgagcttctattaacgccgatgtccacctcgaccttcacggatactttatccctgagaagaagaaggcccagatcacccactag

(19) United States
(12) Patent Application Publication
(10) Pub. No.: US 2020/0277644 A1
(43) Pub. Date: Sep. 3, 2020

(54) PRODUCTION OF TRANS-RETINAL

(71) Applicant: DSM IP ASSETS B.V., Heerlen (NL)

(72) Inventors: Nathalie BALCH, Kaiseraugst (CH); Paul BLOMQUIST, Kaiseraugst (CH); Reed DOTEN, Kaiseraugst (CH); Peter HOUSTON, Kaiseraugst (CH); Ethan LAM, Kaiseraugst (CH); Jenna MCMAHON, Kaiseraugst (CH); Joshua TRUEHEART, Kaiseraugst (CH); Celine VIAROUGE, Kaiseraugst (CH); René Marcel DE JONG, Kaiseraugst (CH)

(21) Appl. No.: 16/649,771

(22) PCT Filed: Sep. 25, 2018
(86) PCT No.: PCT/EP2018/076032

§ 371 Date: Mar. 23, 2020

Related U.S. Application Data
(60) Provisional application No. 62/562,602, filed on Sep. 25, 2017.
(60) Provisional application No. 62/562,601, filed on Sep. 25, 2017.

(51) Int. Cl. C12P 23/00; C12N 9/02

(52) U.S. Cl. CPC C12P 23/00; C12Y 113/11063; C12N 9/0069

(57) ABSTRACT

The present invention is related to a novel enzymatic process for production of vitamin A aldehyde (retinal) via stereoselective conversion of beta-carotene which process includes the use of trans-selective enzymes having activity as beta-carotene oxidases (BCOs), in particular having preference for trans-retinal. 5 Said process is in particular useful for biotechnological production of vitamin A.

The present invention is related to a novel enzymatic process for production of vitamin A aldehyde (retinal) via stereoselective conversion of beta-carotene which process includes the use of trans-selective enzymes having activity as beta-carotene oxidases (BCOs), in particular having preference for trans-retinal. Said process is in particular useful for biotechnological production of vitamin A.

Retinal is an important intermediate/precursor in the process of retinoid production, in particular such as vitamin A production. Retinoids, including vitamin A, are one of very important and indispensable nutrient factors for human beings which have to be supplied via nutrition. Retinoids promote well-being of humans, inter alia in respect of vision, the immune system and growth.

Current chemical production methods for retinoids, including vitamin A and precursors thereof, have some undesirable characteristics such as e.g. high-energy consumption, complicated purification steps and/or undesirable by-products. Therefore, over the past decades, other approaches to manufacture retinoids, including vitamin A and precursors thereof, including microbial conversion steps, which would be more economical as well as ecological, have been investigated.

In general, the biological systems that produce retinoids are industrially intractable and/or produce the compounds at such low levels that commercial scale isolation is not practicable. There are several reasons for this, including instability of the retinoids in such biological systems or the relatively high production of by-products.

Thus, it is an ongoing task to improve the product-specificity and/or productivity of the enzymatic conversion of beta-carotene into vitamin A. Particularly, it is desirable to optimize the selectivity of enzymes involved in conversion of beta-carotene towards production of trans-isoforms, such as e.g. trans-retinal, which are deemed to be the most stable isoform.

Surprisingly, we now could identify so-called trans-cleavage enzymes isolated from various species, i.e. enzymes which are capable of selective conversion of beta-carotene into retinal, in particular trans-retinal, wherein the productivity and/or selectivity of such enzymes toward production of trans-isoforms leading to a retinal mix with product ratios between trans- and cis-isoforms which are at least about 2, preferably wherein the production of trans-isoforms is in the range of at least about 65% based on the total amount of retinoids.

In particular, the present invention is directed to BCOs having the activity of stereoselective oxidizing beta-carotene towards trans-isoforms, such as e.g. trans-retinal, i.e. the conversion of beta-carotene into a retinal mix comprising trans- and cis-retinal, wherein the amount of cis-retinal has been reduced or abolished relative to the amount of trans-retinal, based on the total amount of retinal, leading particularly to percentage of cis-retinal of about 35% and less based on the total amount of retinals.

The invention is preferably directed to a carotenoid-producing host cell, particularly fungal host cell, in particular a retinoid-producing host cell, comprising such selective BCO as defined herein, said host cell producing a retinal mix comprising both cis- and trans-retinal, wherein the percentage of trans-retinal is at least about 65%, preferably 68, 70, 75, 80, 85, 90, 95, 98% or up to 100% based on the total amount of retinal produced by said host cell.

The terms “beta-carotene oxidizing enzyme”, “beta-carotene oxygenase”, “enzyme having beta-carotene oxidizing activity” or “BCO” are used interchangeably herein and refer to enzymes which are capable of catalyzing the conversion of beta-carotene into retinal, in particular wherein the activity towards oxidation of beta-carotene to cis-isoforms, such as e.g. cis-retinal, has been reduced or abolished relative to the activity towards oxidation into trans-isoforms, such as e.g. trans-retinal. Such BCOs are referred herein as stereoselective enzymes, with a preference towards production of trans-isoforms over cis-isoforms.

The terms “conversion”, “oxidation”, “cleavage” in connection with enzymatic catalysis of beta-carotene leading to retinal via action of the described BCOs, i.e. leading to a mix of trans- and cis-isoforms as defined herein, are used interchangeably herein.

As used herein, the terms “stereoselective”, “selective”, “trans-selective” enzyme with regards to BCO are used interchangeably herein. They refer to enzymes, i.e. BCOs as disclosed herein, with increased catalytic activity towards trans-isomers, i.e. increased activity towards catalysis of beta-carotene into trans-retinal. An enzyme according to the present invention is trans-specific, if the percentage of trans-isoforms, such as e.g. trans-retinal, is in the range of at least about 65% based on the total amounts of retinoids produced by such an enzyme or such carotene-producing host cell, particularly fungal host cell, comprising/expressing such enzyme.

As used herein, the term “fungal host cell” includes particularly yeast as host cell, such as e.g. Yarrowia or Saccharomyces.

The stereoselective enzymes as defined herein leading to reduced or abolished production of cis-isoform, in particular cis-retinal, might be introduced into a suitable host cell, i.e. expressed as heterologous enzymes, or might be expressed as endogenous enzymes. They might be obtainable from any carotenoid-producing organism, such as retinoid-producing organism, including plants, animals, algae, fungi or bacteria, preferably fungi, algae, plants, animals.

Compared to the known BCOs, such as e.g. the Drosophila melanogaster BCO according to SEQ ID NO:7, a suitable stereoselective BCO according to the present invention shows an improved product ratio towards production of trans-isoforms, e.g. trans-retinal in the retinal mix comprising trans- and cis-retinal, generated from the conversion of beta-carotene, which is increased by at least about 6% towards trans-isoform compared to the use of the known Drosophila melanogaster BCO sequence (SEQ ID NO:7). Preferably, the amount of trans-retinal in the retinal mix comprising trans- and cis-retinal is increased by at least about 10, 20, 30, 40, 45, 48, 50, 55, 56, 60, 61, 62, 63, 64, or even increased by at least about 70-100%% compared to amount of trans-retinal produced with the Drosophila BCO, i.e. leading to amounts of trans-retinal in the retinal mix in the range of at least about 65 to 90%, at least about 95 or 98% or even up to about 100% trans-retinal.

In one embodiment, the polypeptides having BCO activity as defined herein, preferably stereoselective action towards the formation of trans-retinal, are obtainable from fungi, in particular Dikarya, including but not limited to fungi selected from Ascomycota or Basidiomycota, in particular said polypeptides and/or the genes encoding said BCOs, as defined herein are originated from Fusarium or Ustilago, preferably isolated from F. fujikuroi or U. maydis.

In one preferred embodiment, the polypeptide having stereoselective BCO activity as defined herein, preferably beta-carotene to trans-retinal oxidizing activity with an amount of at least about 65% of trans-retinal compared to cis-retinal based on the total amount of retinal, is selected from a polypeptide with at least 60%, such as e.g. 65, 70, 75, 80, 85, 90, 95, 97, 98, 99% or up to 100% identity to a polypeptide sequence derived from EAK81726, such as e.g. BCO from Ustilago maydis (UmCC01), e.g. polypeptides with at least at least 60%, such as e.g. 65, 70, 75, 80, 85, 90, 95, 97, 98, 99% or up to 100% identity to a polypeptide according to SEQ ID NO:1, including a polypeptide encoded by e.g. a polynucleotide of SEQ ID NO:2.

In one particular preferred embodiment the carotenoid-producing host cell, particularly fungal host cell, comprises a fungal BCO, such as e.g. selected from Ustilago or Fusarium as defined herein, said host cells are grown with gene copy numbers of the BCO below 2 or on low expression promoters, such as particularly 400 base pairs upstream of the Yarrowia lipolytica EN01 gene accession XM_505509.1, resulting in increased output of retinal product due to less nonspecific oxidative activity on precursors and/or cellular components, or other particularly useful promoter elements such as HYPO, HSP, CWP, TPI ENO, ALK (WO2015116781). The skilled person knows how to further modify the respective host cells for optimal activity of fungal BCOs as defined herein.

In one preferred embodiment, the polypeptide having stereoselective BCO activity as defined herein, preferably beta-carotene to trans-retinal oxidizing activity with an amount of at least about 65% of trans-retinal compared to cis-retinal based on the total amount of retinal, is selected from a polypeptide with at least 60%, such as e.g. 65, 70, 75, 80, 85, 90, 95, 97, 98, 99% or up to 100% identity to a polypeptide sequence derived from AJ854252.1, such as e.g. BCO from Fusarium fujikuroi (FfCarX), e.g. polypeptides with at least 60%, such as e.g. 65, 70, 75, 80, 85, 90, 95, 97, 98, 99% or up to 100% identity to a polypeptide according to SEQ ID NO:3, including a polypeptide encoded by e.g. a polynucleotide of SEQ ID NO:4.

In a further embodiment, the polypeptides having stereoselective BCO activity as defined herein, preferably stereoselective action towards the formation of trans-retinal, are obtainable from Eukaryotes, in particular plants, including but not limited to Angiosperms, in particular said polypeptides and/or the genes encoding said BCOs, as defined herein are originated from Crocus, preferably isolated from C. sativus.

In one preferred embodiment, the polypeptide having stereoselective BCO activity as defined herein, preferably beta-carotene to trans-retinal oxidizing activity with an amount of at least about 65% of trans-retinal compared to cis-retinal based on the total amount of retinal, is selected from a polypeptide with at least 60%, such as e.g. 65, 70, 75, 80, 85, 90, 95, 97, 98, 99% or up to 100% identity to a polypeptide derived from sequence Q84K96.1, such as e.g. BCO from Crocus sativus (CsZCO), e.g. polypeptides with at least 60%, such as e.g. 65, 70, 75, 80, 85, 90, 95, 97, 98, 99% or up to 100% identity to a polypeptide according to SEQ ID NO:5, including a polypeptide encoded by e.g. a polynucleotide of SEQ ID NO:6.

In a further embodiment, the polypeptides having stereoselective BCO activity as defined herein, preferably stereoselective action towards the formation of trans-retinal, are obtainable from Eukaryotes, in particular pesces, including but not limited to Actinopterygii, in particular said polypeptides and/or the genes encoding said BCOs, as defined herein are originated from Danio Ictalurus, Esox, or Latimeria preferably isolated from D. rerio, I. punctatus, E. lucius or L. chalumnae.

In one preferred embodiment, the polypeptide having stereoselective BCO activity as defined herein, preferably beta-carotene to trans-retinal oxidizing activity with an amount of at least about 65% of trans-retinal compared to cis-retinal based on the total amount of retinal, is selected from a polypeptide with at least 50%, such as e.g. 55, 60, 65, 70, 75, 80, 85, 90, 93, 95, 97, 98, 99% or up to 100% identity to a polypeptide according to SEQ ID NO:9, 11, 13, 15 or 17 including a polypeptide encoded by e.g. a polynucleotide of SEQ ID NO:10, 12, 14, 16 or 18.

An increase in production of trans-isomers in the retinal mix means an increase of at least about 6% trans-retinal based on the total amount of retinals produced via enzymatic conversion of beta-carotene compared to the amount trans-retinal obtained in a process using the known Drosophila melanogaster (SEQ ID NO:7). This can be achieved by use of a fungal, plant or fish stereoselective BCO as described herein.

“Heterologous expressed” as defined herein means that the gene expressing one of the BCOs as defined herein are introduced into the carotenoid-producing host cell, particularly fungal host cell. Technologies in order to introduce foreign nucleic acid molecules into a cell, such as a carotenoid-producing host cell, particularly fungal host cell, as defined herein, are known in the art. They include the use of promoters and terminators of various strengths and isolators to restrict trans effects on the expression of important genes. Further the advent of synthetic biology has made the use of these techniques routine. A host cell according to the present invention might comprise/express a fungal BCO as disclosed herein, preferably comprising only one copy of a polynucleotide encoding e.g. the fungal BCOs as defined herein, such as e.g. BCO isolated from Ustilago or Fusarium, more preferably BCO from F. fujikuroi or U. maydis, most preferably a BCO with at least 60%, such as e.g. 65, 70, 75, 80, 85, 90, 95, 97, 98, 99% or up to 100% identity to polypeptide according SEQ ID NO: 1 or 2. Alternatively, the fungal BCO might be expressed under the control of a low expression promoter.

Modifications in order to have the host cell as defined herein produce more copies of genes and/or proteins, such as e.g. stereoselective BCOs with selectivity towards formation of trans-retinal as defined herein, may include the use of strong promoters, suitable transcriptional- and/or translational enhancers, or the introduction of one or more gene copies into the carotenoid-producing host cell, particularly fungal cells, leading to increased accumulation of the respective enzymes in a given time. The skilled person knows which techniques to use in dependence of the host cell. The increase or reduction of gene expression can be measured by various methods, such as e.g. Northern, Southern or Western blot technology as known in the art. These technologies are particularly useful for expression of non-fungal BCOs.

The generation of a mutation into nucleic acids or amino acids, i.e. mutagenesis, may be performed in different ways, such as for instance by random or side-directed mutagenesis, physical damage caused by agents such as for instance radiation, chemical treatment, or insertion of a genetic element. The skilled person knows how to introduce mutations.

The BCOs as defined herein might be expressed on a plasmid suitable for expression in the respective host cell, as known by the skilled person.

Thus, the present invention is directed to a carotenoid-producing host cell, particularly fungal host cell, as described herein comprising an expression vector or a polynucleotide encoding BCOs as described herein which has been integrated in the chromosomal DNA of the host cell. Such carotenoid-producing host cell comprising a heterologous polynucleotide either on an expression vector or integrated into the chromosomal DNA encoding BCOs as described herein is called a recombinant host cell. The carotenoid-producing host cell, particularly fungal host cell, might contain one or more copies of a gene encoding the BCOs as defined herein, such as e.g. polypeptides with at least about 60% identity to polypeptides according to SEQ ID NOs:1, 3 or 5, or at least about 50% identity to polypeptides according to SEQ ID NOs:9, 11, 13, 15 or 17, leading to overexpression of such genes encoding the BCOs as defined herein. With regards to fungal BCOs as defined herein, a gene copy of 1 is preferred. The increase of gene expression can be measured by various methods, such as e.g. Northern, Southern or Western blot technology as known in the art.

Based on the sequences as disclosed herein and of the preference for trans-isoforms, i.e. the stereoselective activity, one could easily deduce further suitable genes encoding polypeptides having stereoselective BCO activity as defined herein which could be used for the conversion of beta-carotene into retinal, in particular at least about 65% of trans-retinal compared to cis-retinal based on the total amount of retinal. Thus, the present invention is directed to a method for identification of novel stereoselective BCOs, wherein a polypeptide with at least 60%, such as e.g. 65, 70, 75, 80, 85, 90, 95, 97, 98, 99% or up to 100% identity to polypeptides according to SEQ ID NOs:1, 3 or 5, or at least about 50% identity to polypeptides according to SEQ ID NOs:9, 11, 13, 15 or 17 is used as a probed in a screening process for new stereoselective BCOs with preference for production of trans-isoforms. Any polypeptide having BCO activity might be used for production of retinal from beta-carotene as described herein, as long as the stereoselective action results in at least about 65% trans-retinal compared to the amount of cis-retinal in the produced retinal mix. Thus, a suitable BCO to be used for a process according to the present invention includes an enzyme capable to produce about 35% or less of cis-isoform, such as e.g. about 35% or less cis-retinal, based on the total amount of retinal, from the conversion of beta-carotene.

The present invention is particularly directed to the use of such stereoselective BCOs in a process for production of a retinal mix comprising trans- and cis-retinal, wherein the production of cis-retinal has been reduced or abolished and wherein the production of trans-retinal has been increased, leading to a ratio between trans- and cis-retinal in the retinal mix of at least about 2. The process might be performed with a suitable carotenoid-producing host cell, particularly fungal host cell, expressing said stereoselective BCOs, preferably wherein the genes encoding said BCOs are heterologous expressed, i.e. introduced into said host cells. Retinal, preferably trans-retinal, can be further converted into vitamin A by the action of (known) suitable mechanisms.

Thus, the present invention is directed to a process for decreasing the percentage of cis-retinal in a retinal-mix, or for increasing the percentage of trans-retinal in a retinal mix, wherein the retinal is generated via contacting one of the BCOs as defined herein with beta-carotene, resulting in a retinal-mix with a percentage of at least about 65 to 98% trans-retinal or about 35% or less of cis-retinal. Particularly, said process comprising (a) introducing a nucleic acid molecule encoding one of the stereoselective BCOs as defined herein into a suitable carotenoid-producing host cell, particularly fungal host cell, as defined herein, (b) enzymatic cleavage of beta-carotene into cis-/trans-retinal-mix via action of said expressed stereoselective BCO wherein the percentage of trans retinal in the mix is at least 65% based on the total amount of retinal, and optionally (3) conversion of retinal, preferably trans-retinal, into vitamin A under suitable conditions known to the skilled person.

As used herein, reduction or abolishing the activity towards conversion of beta-carotene into cis-isoforms, e.g. cis-retinal, i.e. improvement of the product ratio towards beta-carotene conversion into trans-isoforms, e.g. trans-retinal, means a product ratio between trans to cis, e.g. trans- to cis-retinal, which is at least about 2:1, such as at least about 3:1, in particular 4:1, 5:1, 6:1, 7:1, 8:1, 9:1, 9.2:1, 9.5:1, 9.8:1 or even up to 10:1, which product ratios are achieved with the stereospecific BCOs as defined herein.

A reduction or abolishment of production of cis-isomers in the retinal mix means a limitation to an amount of about 35% or less cis-retinal based on the total amount of retinals produced via enzymatic conversion of beta-carotene. This can be achieved by the use of a stereoselective BCO as described herein.

As used herein, the term “at least about 65%” with regards to production of trans-isoforms, in particular with regards to production of trans-retinal from conversion of beta-carotene using a BCO as defined herein, means that at least about 65%, such as e.g. 68, 70, 75, 80, 85, 90, 95, 98% or up to 100% of the produced retinal is in the form of trans-retinal. The term “about 35% or less” with regards to production of cis-isoforms, in particular with regards to production of cis-retinal from conversion of beta-carotene using a stereoselective BCO as defined herein, means that about 35% or less, such as e.g. 30, 25, 20, 15, 10, 5, 2 or up to 0% of the produced retinal is in the form of cis-retinal.

The terms “sequence identity”, “% identity” or “sequence homology” are used interchangeable herein. For the purpose of this invention, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, Longden and Bleasby, Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest identity”. If both amino acid sequences which are compared do not differ in any of their amino acids, they are identical or have 100% identity. With regards to enzymes originated from plants as defined herein, the skilled person is aware of the fact that plant-derived enzymes might contain a chloroplast targeting signal which is to be cleaved via specific enzymes, such as e.g. chloroplast processing enzymes (CPEs).

Depending on the host cell, the polynucleotides as defined herein, such as e.g. the polynucleotides encoding a polypeptide according to SEQ ID NOs:1, 3, 5, 7, 9, 11, 13, 15 or 17 might be optimized for expression in the respective host cell. The skilled person knows how to generate such modified polynucleotides. It is understood that the polynucleotides as defined herein also encompass such host-optimized nucleic acid molecules as long as they still express the polypeptide with the respective activities as defined herein.

Thus, in one embodiment, the present invention is directed to a carotenoid-producing host cell, particularly fungal host cell, comprising polynucleotides encoding BCOs as defined herein which are optimized for expression in said host cell, with no impact on growth or expression pattern of the host cell or the enzymes. Particularly, a carotenoid-producing host cell, particularly fungal host cell, is selected from Yarrowia, such as Yarrowia lipolytica, wherein the polynucleotides encoding the BCOs as defined herein are selected from polynucleotides with at least about at least 60%, such as e.g. 65, 70, 75, 80, 85, 90, 92, 95, 97, 98, 99% or up to 100% identity to SEQ ID NOs:2, 4, 6 or at least about 50%, such as e.g. 55, 60, 65, 70, 75, 80, 85, 90, 93, 95, 97, 98, 99% or up to 100% to SEQ ID NOs: 10, 12, 14, 16 or 18.

The BCOs as defined herein also encompass enzymes carrying amino acid substitution(s) which do not alter enzyme activity, i.e. which show the same properties with respect to the wild-type enzyme and catalyze the conversion of beta-carotene into retinal, in particular into an amount of at least about 65% of trans-retinal. Such mutations are also called “silent mutations”, which do not alter the (enzymatic) activity of the enzymes as described herein.

A nucleic acid molecule according to the invention may comprise only a portion or a fragment of the nucleic acid sequence provided by the present invention, such as for instance the sequences as disclosed herein for example a fragment which may be used as a probe or primer or a fragment encoding a portion of a BCO as defined herein. The nucleotide sequence determined from the cloning of the BCO gene allows for the generation of probes and primers designed for use in identifying and/or cloning other homologues from other species. The probe/primer typically comprises substantially purified oligonucleotides which typically comprises a region of nucleotide sequence that hybridizes preferably under highly stringent conditions to at least about 12 or 15, preferably about 18 or 20, more preferably about 22 or 25, even more preferably about 30, 35, 40, 45, 50, 55, 60, 65, or 75 or more consecutive nucleotides of a nucleotide sequence shown in sequences disclosed herein or a fragment or derivative thereof.

A preferred, non-limiting example of such hybridization conditions are hybridization in 6× sodium chloride/sodium citrate (SSC) at about 45° C., followed by one or more washes in 1×SSC, 0.1% SDS at 50° C., preferably at 55° C., more preferably at 60° C. and even more preferably at 65° C.

Highly stringent conditions include, for example, 2 h to 4 days incubation at 42° C. using a digoxigenin (DIG)-labeled DNA probe (prepared by using a DIG labeling system; Roche Diagnostics GmbH, 68298 Mannheim, Germany) in a solution such as DigEasyHyb solution (Roche Diagnostics GmbH) with or without 100 μg/ml salmon sperm DNA, or a solution comprising 50% formamide, 5×SSC (150 mM NaCl, 15 mM trisodium citrate), 0.02% sodium dodecyl sulfate, 0.1% N-lauroylsarcosine, and 2% blocking reagent (Roche Diagnostics GmbH), followed by washing the filters twice for 5 to 15 minutes in 2×SSC and 0.1% SDS at room temperature and then washing twice for 15-30 minutes in 0.5×SSC and 0.1% SDS or 0.1×SSC and 0.1% SDS at 65-68° C.

Expression of the enzymes/polynucleotides encoding one of the stereoselective BCOs as defined herein can be achieved in any host system, including (micro)organisms, which is suitable for carotenoid/retinoid production and which allows expression of the nucleic acids encoding one of the enzymes as disclosed herein, including functional equivalents or derivatives as described herein. Examples of suitable carotenoid/retinoid-producing host (micro)organisms are bacteria, algae, fungi, including yeasts, plant or animal cells. Preferred bacteria are those of the genera Escherichia, such as, for example, Escherichia coli, Streptomyces, Pantoea (Erwinia), Bacillus, Flavobacterium, Synechococcus, Lactobacillus, Corynebacterium, Micrococcus, Mixococcus, Brevibacterium, Bradyrhizobium, Gordonia, Dietzia, Muricauda, Sphingomonas, Synochocystis, Paracoccus, such as, for example, Paracoccus zeaxanthinifaciens. Preferred eukaryotic microorganisms, in particular fungi including yeast, are selected from Saccharomyces, such as Saccharomyces cerevisiae, Aspergillus, such as Aspergillus niger, Pichia, such as Pichia pastoris, Hansenula, such as Hansenula polymorpha, Phycomyces, such as Phycomyces blakesleanus, Mucor, Rhodotorula, Sporobolomyces, Xanthophyllomyces, Phaffia, Blakeslea, such as e.g. Blakeslea trispora, or Yarrowia, such as Yarrowia lipolytica. In particularly preferred is expression in a fungal host cell, such as e.g. Yarrowia or Saccharomyces, or expression in Escherichia, more preferably expression in Yarrowia lipolytica or Saccharomyces cerevisiae.

With regards to the present invention, it is understood that organisms, such as e.g. microorganisms, fungi, algae or plants also include synonyms or basonyms of such species having the same physiological properties, as defined by the International Code of Nomenclature of Prokaryotes or the International Code of Nomenclature for algae, fungi, and plants (Melbourne Code).

As used herein, a carotenoid-producing host cell, particularly fungal host cell, is a host cell, wherein the respective polypeptides are expressed and active in vivo leading to production of carotenoids, e.g. beta-carotene. The genes and methods to generate carotenoid-producing host cells are known in the art, see e.g. WO2006102342. Depending on the carotenoid to be produced, different genes might be involved.

As used herein, a retinoid-producing host cell, particularly fungal host cell, is a host cell wherein, the respective polypeptides are expressed and active in vivo, leading to production of retinoids, e.g. vitamin A and its precursors, via enzymatic conversion of beta-carotene. These polypeptides include the BCOs as defined herein. The genes of the vitamin A pathway and methods to generate retinoid-producing host cells are known in the art. Preferably, the beta-carotene is converted into retinal via action of BCO as defined herein, the retinal is further converted into retinol via action of enzymes having retinol dehydrogenase activity, and the retinol is converted into retinol acetate via action of acetyl-transferase enzymes, such as e.g. ATF1. The retinol acetate might be the retinoid of choice which is isolated from the host cell.

The present invention is directed to a process for production of retinal, in particular trans-isoform of retinal with an amount of at least 65% of trans-retinal, via enzymatic conversion of beta-carotene by the action of a BCO as described herein, wherein the BCOs are preferably heterologous expressed in a suitable host cell under suitable conditions as described herein. The produced retinal, in particular trans-retinal, might be isolated and optionally further purified from the medium and/or host cell. In a further embodiment, retinal, in particular trans-retinal, can be used as precursor in a multi-step process leading to vitamin A. Vitamin A might be isolated and optionally further purified from the medium and/or host cell as known in the art.

The host cell, i.e. microorganism, algae, fungal, animal or plant cell, which is able to express the beta-carotene producing genes, the BCOs as defined herein and/or optionally further genes required for biosynthesis of vitamin A, may be cultured in an aqueous medium supplemented with appropriate nutrients under aerobic or anaerobic conditions and as known by the skilled person for the different host cells. Optionally, such cultivation is in the presence of proteins and/or co-factors involved in transfer of electrons, as defined herein. The cultivation/growth of the host cell may be conducted in batch, fed-batch, semi-continuous or continuous mode. Depending on the host cell, preferably, production of retinoids such as e.g. vitamin A and precursors such as retinal can vary, as it is known to the skilled person. Cultivation and isolation of beta-carotene and retinoid-producing host cells selected from Yarrowia is described in e.g. WO2008042338. With regards to production of retinoids in host cells selected from E. coli, methods are described in e.g. Jang et al, Microbial Cell Factories, 10:95 (2011). Specific methods for production of beta-carotene and retinoids in yeast host cells, such as e.g. Saccharomyces cerevisiae, are disclosed in e.g. WO2014096992.

As used herein, the term “specific activity” or “activity” with regards to enzymes means its catalytic activity, i.e. its ability to catalyze formation of a product from a given substrate. The specific activity defines the amount of substrate consumed and/or product produced in a given time period and per defined amount of protein at a defined temperature. Typically, specific activity is expressed in μmol substrate consumed or product formed per min per mg of protein. Typically, μmol/min is abbreviated by U (=unit). Therefore, the unit definitions for specific activity of μmol/min/(mg of protein) or U/(mg of protein) are used interchangeably throughout this document. An enzyme is active, if it performs its catalytic activity in vivo, i.e. within the host cell as defined herein or within a system in the presence of a suitable substrate. The skilled person knows how to measure enzyme activity, in particular activity of BCOs as defined herein. Analytical methods to evaluate the capability of a suitable BCO as defined herein for trans-retinal production from conversion of beta-carotene are known in the art, such as e.g. described in Example 4 of WO2014096992. In brief, titers of products such as trans-retinal, cis-retinal, beta-carotene and the like can be measured by HPLC.

Retinoids as used herein include beta carotene cleavage products also known as apocarotenoids, including but not limited to retinal, retinolic acid, retinol, retinoic methoxide, retinyl acetate, retinyl esters, 4-keto-retinoids, 3 hydroxy-retinoids or combinations thereof. Biosynthesis of retinoids is described in e.g. WO2008042338.

Retinal as used herein is known under IUPAC name (2E,4E,6E,8E)-3,7-Dimethyl-9-(2,6,6-trimethylcyclohexen-1-yl)nona-2,4,6,8-tetraenal. It is herein interchangeably referred to as retinaldehyde or vitamin A aldehyde and includes both cis- and trans-isoforms, such as e.g. 11-cis retinal, 13-cis retinal, trans-retinal and all-trans retinal. A mixture of cis- and trans-retinal is referred to herein as “retinal mix”, wherein the percentage “at least about 65%” with regards to trans-retinal” or “about 35% or less” with regards to cis-retinal refers to the ratio of trans-retinal to cis-retinal in such retinal mix.

The term “carotenoids” as used herein is well known in the art. It includes long, 40 carbon conjugated isoprenoid polyenes that are formed in nature by the ligation of two 20 carbon geranylgeranyl pyrophosphate molecules. These include but are not limited to phytoene, lycopene, and carotene, such as e.g. beta-carotene, which can be oxidized on the 4-keto position or 3-hydroxy position to yield canthaxanthin, zeaxanthin, or astaxanthin. Biosynthesis of carotenoids is described in e.g. WO2006102342.

Vitamin A as used herein may be any chemical form of vitamin A found in aqueous solutions, such as for instance undissociated, in its free acid form or dissociated as an anion. The term as used herein includes all precursors or intermediates in the biotechnological vitamin A pathway. It also includes vitamin A acetate.

In particular, the present invention features the present embodiments:
A carotenoid-producing host cell, particularly fungal host cell, comprising a stereoselective beta-carotene oxidizing enzyme (BCO), said host cell producing a retinal mix comprising cis- and trans-retinal, wherein the percentage of trans-retinal in the mix is at least about 65%, preferably 68, 70, 75, 80, 85, 90, 95, 98% or up to 100% produced by said host cell.
The carotenoid-producing host cell, particularly fungal host cell, as above and defined herein, wherein the percentage of trans-retinal in the retinal mix comprising trans- and cis-retinal is in the range of about at least 65 to 98%, preferably about at least 65 to 95%, more preferably at least about 65 to 90% based on the total amount of retinal produced by said host cell.
The carotenoid-producing host cell, particularly fungal host cell, as above and defined herein, comprising a heterologous stereoselective BCO.
The carotenoid-producing host cell as above and defined herein, wherein the host cell is selected from plants, fungi, algae or microorganisms, preferably selected from fungi including yeast, more preferably from Saccharomyces, Aspergillus, Pichia, Hansenula, Phycomyces, Mucor, Rhodotorula, Sporobolomyces, Xanthophyllomyces, Phaffia, Blakeslea or Yarrowia, even more preferably from Yarrowia lipolytica or Saccharomyces cerevisiae.
The carotenoid-producing host cell as above and defined herein, wherein the host cell is selected from plants, fungi, algae or microorganisms, preferably selected from Escherichia, Streptomyces, Pantoea, Bacillus, Flavobacterium, Synechococcus, Lactobacillus, Corynebacterium, Micrococcus, Mixococcus, Brevibacterium, Bradyrhizobium, Gordonia, Dietzia, Muricauda, Sphingomonas, Synochocystis or Paracoccus.
The carotenoid-producing host cell, particularly fungal host cell, as above and defined herein, wherein the BCO is selected from fungi, plants or fish, preferably selected from Fusarium, Ustilago, Crocus, Danio or Ictalurus, more preferably selected from Fusarium fujikuroi, Ustilago maydis, Crocus sativus, Danio rerio, Ictalurus punctatus, Esox lucius, Latimeria chalumnae, most preferably selected from a polypeptide with at least about 60% identity to a polypeptide according to SEQ ID NOs:1, 2 or 3, or with at least about 50% identity to a polypeptide according to a polypeptide according to SEQ ID NOs:9, 11, 13, 15 or 17.
The carotenoid-producing host cell, particularly fungal host cell, as above and defined herein, wherein the trans-retinal is further converted into vitamin A.
A process for production of a retinal mix comprising trans- and cis-retinal via enzymatic activity of a stereoselective BCO as defined herein, comprising contacting beta-carotene with said BCO, wherein the ratio of trans-retinal to cis-retinal in the retinal mix is at least about 2:1.
A process for decreasing the amount of cis-retinal produced from enzymatic cleavage of beta-carotene, said process comprising contacting beta-carotene with a stereoselective BCO as defined herein, wherein the amount of cis-retinal in the retinal mix resulting from cleavage of beta-carotene is in the range of about 35% or less based on the total amount of retinal.
A process for increasing the amount of trans-retinal produced from enzymatic cleavage of beta-carotene, said process comprising contacting beta-carotene with a stereoselective BCO as defined herein, wherein the amount of trans-retinal in the retinal mix is in the range of at least about 65 to 98% based on the total amount of retinal.
A process as above and defined herein using a carotenoid-producing host cell, particularly fungal host cell, as defined herein comprising a stereoselective beta-carotene oxidizing enzyme (BCO), said host cell producing a retinal mix comprising cis- and trans-retinal, wherein the percentage of trans-retinal is at least about 65%, preferably 68, 70, 75, 80, 85, 90, 95, 98% or up to 100% based on the total amount of retinal produced by said host cell.
A process for production of vitamin A comprising the steps of:
   (a) introducing a nucleic acid molecule encoding a stereoselective BCO as defined herein, into a suitable carotene-producing host cell, particularly fungal host cell,
   (b) enzymatic conversion of beta-carotene into a retinal mix as defined herein comprising cis- and trans-retinal, wherein the percentage of trans-retinal is at least about 65% based on the total amount of retinal,
   (c) conversion of trans-retinal into vitamin A under suitable culture conditions.
Use of a carotenoid-producing host cell, particularly fungal host cell, as above and defined herein for production of a retinal mix comprising trans- and cis-retinal in a ratio of 2:1, wherein said host cell is expressing a heterologous BCO with stereoselectivity towards production of trans-isoforms.

The following examples are illustrative only and are not intended to limit the scope of the invention in any way. The contents of all references, patent applications, patents and published patent applications, cited throughout this application are hereby incorporated by reference, in particular WO2006102342, WO2008042338 or WO2014096992.

### EXAMPLES

### Example 1: General Methods, Strains and Plasmids

All basic molecular biology and DNA manipulation procedures described herein are generally performed according to Sambrook et al. (eds.), Molecular Cloning: A Laboratory Manual. Cold Spring Harbor Laboratory Press: New York (1989) or Ausubel et al. (eds). Current Protocols in Molecular Biology. Wiley: New York (1998).

### Shake Plate Assay.

Typically, 800 μl of 0.075% Yeast extract, 0.25% peptone (0.25×YP) is inoculated with 10 μl of freshly grown Yarrowia and overlaid with 200 μl of Drakeol 5 mineral oil carbon source 5% corn oil in mineral oil and/or 5% in glucose in aqueous phase. Transformants were grown in 24 well plates (Multitron, 30° C., 800 RPM) in YPD media with 20% dodecane for 4 days. The mineral oil fraction was removed from the shake plate wells and analyzed by HPLC on a normal phase column, with a photo-diode array detector.

### DNA Transformation.

Strains are transformed by overnight growth on YPD plate media 50 μl of cells is scraped from a plate and transformed by incubation in 500 μl with 1 μg transforming DNA, typically linear DNA for integrative transformation, 40% PEG 3550 MW, 100 mM lithium acetate, 50 mM Dithiothreitol, 5 mM Tris-Cl pH 8.0, 0.5 mM EDTA for 60 minutes at 40° C. and plated directly to selective media or in the case of dominant antibiotic marker selection the cells are out grown on YPD liquid media for 4 hours at 30° C. before plating on the selective media.

### DNA Molecular Biology.

Genes were synthesized with NheI and MluI ends in pUC57 vector. Typically, the genes were subcloned to the MB5082 ‘URA3’, MB6157 HygR, and MB8327 NatR vectors for marker selection in Yarrowia lipolytica transformations, as in WO2016172282. For clean gene insertion by random nonhomologous end joining of the gene and marker HindIII/XbaI (MB5082) or PvuII (MB6157 and MB8327), respectively purified by gel electrophoresis and Qiagen gel purification column.

### Plasmid List.

Plasmid, strains and codon-optimized sequences to be used are listed in Table 1, 2 and the sequence listing. Nucleotide sequence ID NOs:2, 4, 6, 8, 10, 12, 14, 16, 18 are codon optimized for expression in Yarrowia.

TABLE 1
list of plasmids used for construction of the strains
carrying the heterologous BCO-genes. The sequence ID
NOs refer to the inserts. For more details, see text.

| | | | SEQ ID NO: |
|---|---|---|---|
| MB plasmid | Backbone MB | Insert | (aa/nt) |
| 8457 | 5082 | UmCCO1 | 1/2 |
| 8456 | 5082 | FfCarX | 3/4 |
| 6703 | 5082 | CsZCO | 5/6 |
| 6702 | 5082 | DmNinaB | 7/8 |
| 9068 | 5082 | DrBCO | 9/10 |
| 9279 | 5082 | DrBCO-TPI | 11/12 |
| 9123 | 5082 | IpBCO | 13/14 |
| 9121 | 5082 | ElBCO | 15/16 |
| 9126 | 5082 | LcBCO | 17/18 |

TABLE 2
list of Yarrowia strains used for production of retinoids carrying
the heterologous BCO genes. For more details, see text.

| ML strain | Description | First described in |
|---|---|---|
| 7788 | Carotene strain | WO2016172282 |
| 15710 | ML7788 transformed with | WO2016172282 |
| | MB7311 -Mucor CarG | |
| 17544 | ML15710 cured of URA3 by | here |
| | FOA and HygR by Cre/lox | |
| 17767 | ML17544 transformed with | here |
| | MB6072 DmBCO-URA3 | |
| | and MB6732 SbATF1-HygR | |
| | and cured of markers | |
| 17978 | ML17968 transformed with | here |
| | MB8200 FfRDH-URA3 | |
| | and cured of markers | |

### Normal Phase Retinol Method.

A Waters 1525 binary pump attached to a Waters 717 auto sampler were used to inject samples. A Phenomenex Luna 3μ Silica (2), 150×4.6 mm with a security silica guard column kit was used to resolve retinoids. The mobile phase consists of either, 1000 mL hexane, 30 mL isopropanol, and 0.1 mL acetic acid for astaxanthin related compounds, or 1000 mL hexane, 60 mL isopropanol, and 0.1 mL acetic acid for zeaxanthin related compounds. The flow rate for each is 0.6 mL per minute. Column temperature is ambient. The injection volume is 20 μL. The detector is a photodiode array detector collecting from 210 to 600 nm. Analytes were detected according to Table 3.

TABLE 3
list of analytes using normal phase retinol method.
The addition of all added intermediates gives the amount
of total retinoids. For more details, see text.

| | Retention time | Lambda max |
|---|---|---|
| Intermediates | [min] | [nm] |
| 11-cis-dihydro-retinol | 7.1 | 293 |
| 11-cis-retinal | 4 | 364 |
| 11-cis-retinol | 8.6 | 318 |
| 13-cis-retinal | 4.1 | 364 |
| dihydro-retinol | 9.2 | 292 |
| retinyl-acetate | 3.5 | 326 |
| retinyl-ester | 3 | 325 |
| trans-retinal | 4.7 | 376 |
| trans-retinol | 10.5 | 325 |

### Sample Preparation.

Samples were prepared by various methods depending on the conditions. For whole broth or washed broth samples the broth was placed in a Precellys® tube weighed and mobile phase was added, the samples were processed in a Precellys® homogenizer (Bertin Corp, Rockville, Md., USA) on the highest setting 3× according to the manufactures directions. In the washed broth the samples were spun in a 1.7 ml tube in a microfuge at 10000 rpm for 1 minute, the broth decanted, 1 ml water added mixed pelleted and decanted and brought up to the original volume the mixture was pelleted again and brought up in appropriate amount of mobile phase and processed by Precellys® bead beating. For analysis of mineral oil fraction, the sample was spun at 4000 RPM for 10 minutes and the oil was decanted off the top by positive displacement pipet (Eppendorf, Hauppauge, N.Y., USA) and diluted into mobile phase mixed by vortexing and measured for retinoid concentration by HPLC analysis.

### Fermentation Conditions.

Fermentations were identical to the previously described conditions using mineral oil overlay and stirred tank that was corn oil fed in a bench top reactor with 0.5 L to 5 L total volume (see WO2016172282). Generally, the same results were observed with a fed batch stirred tank reactor with an increased productivity demonstrating the utility of the system for the production of retinoids.

### Example 2: Production of Trans-Retinal in Yarrowia lipolytica

Typically, a beta carotene strain ML17544 was transformed with purified linear DNA fragment by HindII and XbaI mediated restriction endonucleotide cleavage of beta carotene oxidase (BCO) containing codon optimized fragments linked to a URA3 nutritional marker. Transforming DNA were derived from MB6702 Drosophila NinaB BCO gene, MB6703 Crocus BCO gene, MB8456 Fusarium BCO gene, MB8457 Ustilago BCO gene, and MB6098 Dario BCO gene, whereby the codon-optimized sequences (SEQ ID NOs:2, 4, 6, 8, 10, 12) had been used. The genes were then grown screening 6-8 isolates in a shake plate analysis, and isolates that performed well were run in a fed batch stirred tank reaction for 8-10 days. Detection of cis- and trans-retinal was made by HPLC using standard parameters as described in WO2014096992, but calibrated with purified standards for the retinoid analytes. The amount of trans-retinal in the retinal mix could be increased to 90% (using the Crocus BCO), 95% (using the Fusarium BCO), 98% (using the Ustilago BCO) and 98% (using Dario BCO), respectively. A comparison with the BCO from Drosophila melanogaster (SEQ ID NO:7) resulted in only 61% of trans-retinal based on the total amount of retinal (see Table 4).

TABLE 4
Retinal production in Yarrowia as enhanced by action of
heterologous BCOs. “% trans” means percentage of
trans-retinal in the mix of retinoids. For more details, see text.

| | BCO | % | % | ML | MB |
|---|---|---|---|---|---|
| Organism | gene | trans- | retinoids/DCW | strain | plasmid |
| Drosophila | DmNinB | 61 | 14 | 17544 | 6702 |
| Ustilago | UmCCO1 | 98 | 8 | 17544 | 8457 |
| Fusarium | FfCarX | 95 | 5 | 17544 | 8456 |
| Crocus | ZsZCO | 90 | 0.01 | 17544 | 6703 |
| Dario | DrBCO | 98 | 6 | 17544 | 9068 |
| Dario | DrBCO-TPI | 98 | 6 | 17544 | 9279 |
| Ictalurus | IpBCO | 98 | 5 | 17544 | 9123 |
| Esox | ElBCO | 98 | 3 | 17544 | 9121 |
| Latimeria | LcBCO | 98 | 2 | 17544 | 9126 |

### Example 3: Production of Trans-Retinal in Saccharomyces cerevisiae

Typically, a beta carotene strain is transformed with heterologous genes encoding for enzymes such as geranylgeranyl synthase, phytoene synthase, lycopene synthase, lycopene cyclase constructed that is producing beta carotene according to standard methods as known in the art (such as e.g. as described in US20160130628 or WO2009126890). Further, when transformed with beta-carotene oxidase genes as described herein retinal can be produced. Optionally, when transformed with retinol dehydrogenase, then retinol can be produced. The retinol can optionally be acetylated by transformation with genes encoding alcohol acetyl transferases. Optionally, the endogenous retinol acylating genes can be deleted. Further, optionally the enzymes can be selected to produce and acetylate the trans form of retinol to yield all trans retinyl acetate, and long chain esters of trans retinol, respectively. With this approach, similar results regarding specificity for trans-retinal as described herein with Yarrowia lipolytica as host are obtained.

### Example 4: Optimization of Trans-Retinal Production Using Fungal BCOs

Typically, the Ustilago BCO was codon optimized for Yarrowia lipolytica and subcloned using MluI/NheI into vectors in Table 5 below and examined for activity. These plasmids were then transformed into the carotene producing strain MB17544, a lycopene producing strain, MB14925 (erg9::ura3 car8 HMG-tm GGS carRP(E78G) alk1D alk2D) and a phytoene producing strain, MB7206(erg9::ura3bart car8 HMG GGS ura3 ade1) (see Table 5). Surprisingly, there was an optimal activity and we could show that there was an increased production of retinol from a lower activity promoters ALK1, and ACT1. We also observed decreased attenuation of the precursors in the lycopene and phytoene strains.

TABLE 5
list of plasmids used for construction of
the strains. For more details, see text.

| MB plasmid | gene | description |
|---|---|---|
| 6222 | ENO | enolase |
| 6224 | CWP | cell wall protein |
| 6226 | TPI | triose phospate |
| | | isomerase |
| 6228 | GAPDH | glycerol phosphate |
| | | dehydrogenase |
| 6230 | ACT | actin |
| 7311 | ALK | alkane assimilating |
| 6655 | HYPO | Hypothetical |
| 6674 | HSP | Heat shock protein |

1. A carotenoid-producing host cell comprising a stereoselective beta-carotene oxidizing enzyme (BCO), said host cell producing a retinal mix comprising cis- and trans-retinal, wherein the percentage of trans-retinal in the mix is at least about 65%, preferably 68, 70, 75, 80, 85, 90, 95, 98% or up to 100% produced by said host cell.

2. The carotenoid-producing host cell of claim 1, wherein the percentage of trans-retinal in the retinal mix comprising trans- and cis-retinal is in the range of about at least 65 to 98%, preferably about at least 65 to 95%, more preferably at least about 65 to 90% based on the total amount of retinal produced by said host cell.

3. The carotenoid-producing host cell according to claim 1 comprising a heterologous stereoselective BCO.

4. The carotenoid-producing host cell according to claim 1, wherein the BCO is selected from fungi, plants or animal, preferably selected from Fusarium, Ustilago, Crocus, Drosophila, Danio, Ictalurus, Esox, Latimeria, more preferably selected from Fusarium fujikuroi, Ustilago maydis, Crocus sativus, Drosophila melanogaster, Danio rerio, Ictalurus punctatus, Esox lucius, Latimeria chalumnae.

5. The carotenoid-producing host cell according to claim 4, wherein the BCO is selected from a polypeptide with at least about 60% identity to a polypeptide according to sequences known from the database such as EAK81726, AJ854252, Q84K96.1, or with at least 50% identity to a polypeptide according to sequence known from the database as Q90WH4.

6. The carotenoid-producing host cell according to claim 5, expressing a polynucleotide encoding a polypeptide with at least about 60% identity to a polypeptide according to SEQ ID NOs:1, 3, 5 or 7 or a polypeptide with at least about 50% identity to a polypeptide sequence according to SEQ ID NOs:9, 11, 13, 15 or 17.

7. The carotenoid-producing host cell according to claim 1, wherein the host cell is selected from plants, fungi, algae or microorganisms, such as selected from the group consisting of Escherichia, Streptomyces, Pantoea, Bacillus, Flavobacterium, Synechococcus, Lactobacillus, Corynebacterium, Micrococcus, Mixococcus, Brevibacterium, Bradyrhizobium, Gordonia, Dietzia, Muricauda, Sphingomonas, Synochocystis, Paracoccus, Saccharomyces, Aspergillus, Pichia, Hansenula, Phycomyces, Mucor, Rhodotorula, Sporobolomyces, Xanthophyllomyces, Phaffia, and Blakeslea, preferably selected from fungi including yeast, more preferably selected from the group consisting of Saccharomyces, Aspergillus, Pichia, Hansenula, Phycomyces, Mucor, Rhodotorula, Sporobolomyces, Xanthophyllomyces, Phaffia, Blakeslea and Yarrowia, most preferably from Yarrowia lipolytica or Saccharomyces cerevisiae.

8. The carotenoid-producing host cell according to claim 1, wherein the trans-retinal is further converted into vitamin A.

9. A process for production of a retinal mix comprising trans- and cis-retinal via enzymatic activity of a stereoselective BCO, comprising contacting beta-carotene with said BCO, wherein the ratio of trans-retinal to cis-retinal in the retinal mix is at least about 2:1.

10. A process for decreasing the amount of cis-retinal produced from enzymatic cleavage of beta-carotene, said process comprising contacting beta-carotene with a stereoselective BCO, wherein the amount of cis-retinal in the retinal mix resulting from cleavage of beta-carotene is in the range of about 35% or less based on the total amount of retinal.

11. A process for increasing the amount of trans-retinal produced from enzymatic cleavage of beta-carotene, said process comprising contacting beta-carotene with a stereoselective BCO, wherein the amount of trans-retinal in the retinal mix is in the range of at least about 65 to 98% based on the total amount of retinal.

12. A process according to claim 9 using the carotenoid-producing host cell.

13. A process for production of vitamin A comprising the steps of:
(a) introducing a nucleic acid molecule encoding a stereoselective BCO, into a suitable carotene-producing host cell,
(b) enzymatic conversion of beta-carotene into a retinal mix comprising cis- and trans-retinal, wherein the percentage of trans-retinal is at least about 65% based on the total amount of retinal,
(c) conversion of trans-retinal into vitamin A under suitable culture conditions.

14. Use of a carotenoid-producing host cell according to claim 1 for production of a retinal mix comprising trans- and cis-retinal in a ratio of 2:1, wherein said host cell expressing a heterologous BCO with stereoselectivity towards production of trans-isoforms.
